(19) United States
(12) Patent Application Publication
(10) Pub. No.: US 2025/0387128 A1
(43) Pub. Date: Dec. 25, 2025

(54) INTRAVASCULAR DEVICE

(71) Applicant: VentureMed Group, Inc., Plymouth, MN (US)

(72) Inventors: John P. Pigott, Sylvania, OH (US); Jenny Zeroni, Plymouth, MN (US); Adam Tschida, Brooklyn Park, MN (US)

(21) Appl. No.: 19/311,712

(22) Filed: Aug. 27, 2025

Related U.S. Application Data
(63) Continuation of application No. 17/702,336, filed on Mar. 23, 2022, now Pat. No. 12,408,932.

(51) Int. Cl. A61B 17/22; A61B 17/00

(52) U.S. Cl. CPC A61B 17/22; A61B 17/00234; A61B 2017/00199; A61B 2017/00336; A61B 2017/22001; A61B 2017/22048; A61B 2017/22051; A61B 2562/0247; A61B 2562/0261; A61B 2562/04

(57) ABSTRACT

An intravascular device and related methods are disclosed. In an exemplary embodiment, the device includes an expandable portion, which includes struts selectively movable between a first position and a second position where the struts are moved outward. Tissue modification element(s) are each provided at a respective strut. A spring is interposed between the expandable portion and a tip member which is spaced apart from a distal end of the expandable portion. In another exemplary embodiments, the device includes the expandable portion and the tissue modification element(s), and a balloon located between the struts, which when inflated, moves the struts into the second position.

### CROSS-REFERENCE TO RELATED APPLICATIONS

This application is a continuation of U.S. application Ser. No. 17/702,336 filed Mar. 23, 2022, the disclosure of which are hereby incorporated by reference as if fully restated herein.

### FIELD OF THE INVENTION

These disclosures relate, in general, to intravascular devices, such as can be used during minimally invasive surgical procedures. In particular, these disclosures relate to an intravascular device having feedback elements.

### BACKGROUND AND SUMMARY OF THE INVENTION

Atherosclerosis is a chronic condition in which atheromatous plaque accumulates on the inner walls of a blood vessel. As a result, the blood vessel walls can become inflamed and, over time, may harden to form atherosclerotic lesions that cause a narrowing of the vessel lumen. In severe cases, the atherosclerotic lesions can rupture and induce the formation of thrombus (i.e., blood clots), which can prevent blood flow through the narrowed vessel lumen. Certain such areas can be fully or partially blocked over a distance of the blood vessel. In the case of peripheral artery disease, by way of non-limiting example, these areas tend to be less obstructed (e.g., have a lower level of luminal obstruction) but often have some level of obstruction over a longer length of blood vessel. In other cases, the blockages are relatively severe, but located over a shorter distance.

Fistulas are another example of blood vessels that may become partially or wholly blocked over time. For example, without limitation, AV fistulas are often created for dialysis treatment to provide an access point which may be accessed several times a week without collapsing the vessels. These fistulas may be created from a person's own blood vessels, grafts, or artificial implants, to name a few examples. AV fistulas are known to experience stenosis, which can cause damage to, or failure of, the AV fistula. The mechanism and type of stenosis is generally different from that of certain other blood vessel stenosis (e.g., plaque accumulation). For example, without limitation, the stenosed regions may comprise fibrotic tissue.

There are known procedures and devices for treating or otherwise reducing the risks associated with atherosclerosis or other wholly or partially blocked blood vessels. For example, angioplasty is a procedure in which a balloon catheter is inserted into a narrowed region of the vessel lumen via a delivery catheter. The balloon catheter includes a flexible tube having an inflatable balloon at an end thereof. Once positioned in the narrowed region, the balloon is inflated in order to dilate the narrowed vessel lumen. The pressure in the balloon is generally sufficient to compress the accumulated tissue.

However, certain medical interventions can, in some cases, cause dissection in blood vessels. Dissection may include separation between the plates or layers making up the blood vessel and may occur as a result of the compressive forces exerted against the blood vessel from angioplasty, for example. Low grade dissection is generally considered medically acceptable, but larger grade dissection can have undesirable medical effects.

Certain intravascular devices for scoring atherosclerotic lesions are known. Such devices may be used to fragment atherosclerotic lesions, such to improve luminal gain and/or facilitate fragmentation of the atherosclerotic material during a subsequent angioplasty procedure. Examples of such known devices include those provided in U.S. Pat. No. 9,615,848 issued Apr. 11, 2017, US Pub. No. 2021/0220008 published Jul. 22, 2021, the disclosures of which are hereby incorporated by reference as if fully restated herein (hereinafter collectively also the “Prior Disclosures”).

The accumulated tissue in blood vessels does not typically take on a uniform hardness, making controlling and anticipating possible dissection difficult. Some accumulated tissue may be calcified, requiring relatively high pressure for scoring, angioplasty, or other treatment devices to effectuate particular medical effects, such as adequate or desirable luminal gain. Relatively higher pressures may be acceptable in such cases based on the need for the particular medical effect and/or because the pressure is largely transferred to the accumulated tissue rather than the blood vessel or surrounding tissue. Other accumulated tissue may be relatively soft, requiring relatively lower pressure for scoring, angioplasty, or other treatment devices to effectuate the same particular medical effects. Relatively lower pressures may be desirable in such cases because the lower pressures may still provide adequate medical effects and/or because more of the pressure is transferred to the blood vessel wall.

Intravascular devices and systems having feedback elements which, for example without limitation, indicate characteristics of the accumulated tissue (e.g., hardness) and/or forces exerted by the intravascular device (e.g., pressure) are disclosed herein along with methods of utilizing the same. The intravascular devices may comprise some or all of the same or similar components as those provided in the Prior Disclosures, by way of exemplary embodiment without limitation. In exemplary embodiments, without limitation, the intravascular devices may comprise an expandable portion comprising a number of struts configured to be selectively moved between a collapsed position and an expanded position, such as by way of sliding movement of an inner sleeve. The inner sleeve may extend within a catheter tube which extends to a handle assembly, such as within a sheath. The inner sleeve may be connected to a distal end of the struts and/or a tip member at a distal end, and to a control element at the handle assembly at a proximal end. The inner sleeve may accommodate a guide wire. The intravascular devices may alternatively, or additionally, comprise a balloon for moving the struts between the open and closed positions.

One or more feedback devices may be provided at exterior surfaces of said intravascular devices. The feedback devices may comprise devices configured to detect hardness, such as but not limited to durometers, sclerometers, indenters, scleroscopes, pressure sensors, combinations thereof, or the like. Alternatively, or additionally, the feedback devices may comprise devices configured to measure pressure, movement, forces, combinations thereof, or the like, such as but not limited to pressure sensors, strain gauges, accelerometers, combinations thereof, or the like. A single or multiple such feedback devices of the same or different type may be provided at each intravascular device. The feedback devices may be located along one or more struts of the expandable portion in exemplary embodiments.

The feedback devices may be in wired or wireless electronic communication with one or more feedback displays, which may be provided at the intravascular device, or remote therefrom. The feedback displays may be configured to provide feedback data, such as hardness measurements, pressure measurements, force measurements, expansion measurements, combinations thereof, or the like, in substantially real-time or historically from the feedback devices.

For example, without limitation, the feedback devices may be provided on a ring or jacket which surrounds at least part of an outer surface of an expandable portion of the intravascular device. The ring or jacket may surround part or all of an outer surface of a balloon and/or one or more struts forming the expandable portion in exemplary embodiments without limitation. In other exemplary embodiments, without limitation, the feedback devices may be provided along an outer surface of, or integrated with, a balloon or the struts without the need for a separate ring or jacket. While multiple feedback devices are discussed in some places, a single feedback device for each intravascular device may be utilized. Where the intravascular device comprises one or more incising elements, at least one of the feedback devices may, alternatively or additionally, be positioned adjacent to one or more of the incising element(s), and/or at another one of the struts not comprising the incising element, though any location may be utilized. Alternatively, or additionally, feedback devices may be provided at common components which connect multiple ones of the struts, tip members, limiters, combinations thereof, or the like.

The user may monitor the feedback data and adjust expansion of the expandable portion accordingly. For example, without limitation, where harder atherosclerotic material or other tissue is encountered, pressure exerted by the expandable portion may be increased to a relatively higher level. As another example, again without limitation, where softer atherosclerotic material or other tissue is encountered, pressure exerted by the expandable portion may be decreased to a relatively lower level. In other exemplary embodiments, pressure exerted by the expandable portion may be adjusted to one or more benchmarks, thresholds, ranges, or the like which are predetermined to be acceptable or desirable. Such benchmarks, thresholds, ranges, or the like may be those known to result in minimal or acceptable levels of dissection, provide acceptable, desirable, or particular medical effects, combinations thereof, or the like. Such benchmarks, thresholds, ranges, or the like may be specific to hardness levels of the atherosclerotic material, the tissue encountered, the location of the treatment area, combinations thereof, or the like, though such is not required. In this way, chances of dissection or other undesirable effects may be minimized.

Expansion of the expandable portion may be accomplished by way of one or more control devices, which may be located on a handle subassembly of the intravascular devices, though such is not required. Adjustments to the control member may be accomplished manually by the user or may be automatic, such as based on one or more criteria set by the user. For example, without limitation, the one or more control devices may be configured to accept user input regarding desired expansion of the expandable portion, exertion of a particular pressure, treatment of a particular type, location, and/or kind of atherosclerotic material or other tissue (e.g., by hardness level, tissue type), location of treatment area, patient information (e.g., age, weight, risk factors, etc.), combinations thereof, or the like.

Various aspects of this invention will become apparent to those skilled in the art from the following detailed description of the preferred embodiments, when read in light of the accompanying drawings.

### BRIEF DESCRIPTION OF THE DRAWINGS

In addition to the features mentioned above, other aspects of the present invention will be readily apparent from the following descriptions of the drawings and exemplary embodiments, wherein like reference numerals across the several views refer to identical or equivalent features, and wherein:

FIG. 1 is plan view of an exemplary intravascular device with feedback elements and related system, also indicating detail A;

FIG. 2 is a detailed perspective view of an exemplary embodiment of an expandable portion of detail A of FIG. 1, also indicating detail B;

FIG. 3 is a detailed perspective view of detail B of FIG. 2;

FIG. 4 is a detailed perspective view of another exemplary embodiment of an expandable portion of detail A of FIG. 1 with certain components not illustrated for clarity;

FIG. 5 is a detailed perspective view of another exemplary embodiment of an expandable portion of detail A of FIG. 1; and

FIG. 6 is a flow chart with an exemplary method for using the system and devices of FIGS. 1-5.

### DETAILED DESCRIPTION OF THE PREFERRED EMBODIMENTS

Various embodiments of the present invention will now be described in detail with reference to the accompanying drawings. In the following description, specific details such as detailed configuration and components are merely provided to assist the overall understanding of these embodiments of the present invention. Therefore, it should be apparent to those skilled in the art that various changes and modifications of the embodiments described herein can be made without departing from the scope and spirit of the present invention. In addition, descriptions of well-known functions and constructions are omitted for clarity and conciseness.

Embodiments of the invention are described herein with reference to illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

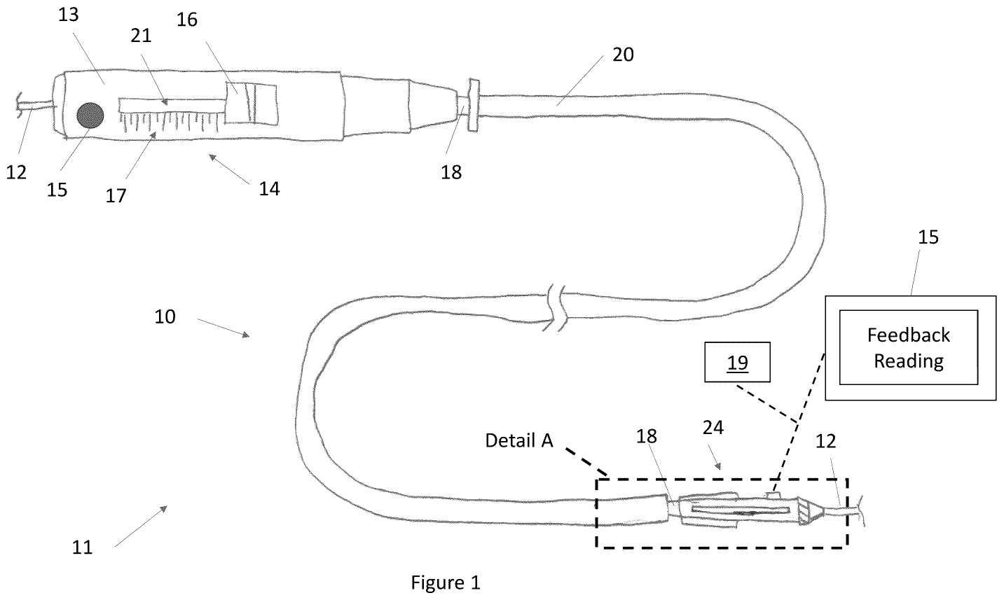

FIG. 1 illustrates a system 11 comprising an intravascular device 10 with feedback elements 34. The intravascular device 10 may comprise some or all of the components of the Prior Disclosures, the contents of which are hereby incorporated by reference as if fully restated herein.

The intravascular device 10 may comprise a handle subassembly 14. The handle subassembly 14 may comprise a housing 13. The handle subassembly 14 may be configured to accommodate a guide wire 12 passing through some or all of the handle subassembly 14.

The handle subassembly 14 may comprise one or more control elements 16. The control element(s) 16 in exemplary embodiments, without limitation, may comprise one or more levers, sliders, dials, knobs, buttons, motors, relays, touch pads, electronic controls, combinations thereof, or the like which are moveable or otherwise actuatable to operate an expandable portion 24 or other components of the device 10.

A catheter tube 18 may extend from the handle subassembly 14 to the expandable portion 24. The catheter tube 18 may be attached to, or extend within, the handle assembly 14.

A sheath 20 may be provided, though such is not necessarily required. Some or all of the catheter tube 18 may extend through the sheath 20. The sheath 20 may be connected to the handle subassembly 14 or be separate therefrom.

An inner sleeve 26 may extend within the catheter tube 18. The inner sleeve 26 may be configured for sliding movement within the catheter tube 18. The inner sleeve 26 may be connected, directly or indirectly, to one or more of the control element(s) 16, in exemplary embodiments without limitation, such that sliding or other actuation of the control element(s) 16 is translated to corresponding movement of the inner sleeve 26.

The catheter tube 18, the inner sleeve 26, the sheath 20, and/or the guide wire 12 may comprise sufficiently flexible material to permit navigation of sinuous blood vessel within a patient's vascular system. The inner sleeve 26 may comprise a tube or other hollow member, though in other exemplary embodiments the inner sleeve 26 may comprise one or more solid members of any size or shape, such as but not limited to a wire, pushrod, linkage, combinations thereof, or the like. In exemplary embodiments, without limitation, the entire inner sleeve 26 and at least a portion of the handle subassembly 14 may be configured to accommodate the guide wire 12. In other exemplary embodiments, only the expandable portion 24, or a portion thereof, may be configured to accommodate the guide wire 12. For example, without limitation, a distal portion of the inner sleeve 26 may comprise one or more holes, slits, or the like for allowing the guide wire 12 to enter and exit a portion of the expandable portion 24.

One or more indicators 17 may be provided, such as at the handle subassembly 14, for indicating status of the expandable portion 24. The indicators 17 may comprise marking on the housing 13 (e.g., for marking location of one or more of the control element(s) 16 relative to the housing), electronic displays, gauge, lights, combinations thereof, or the like.

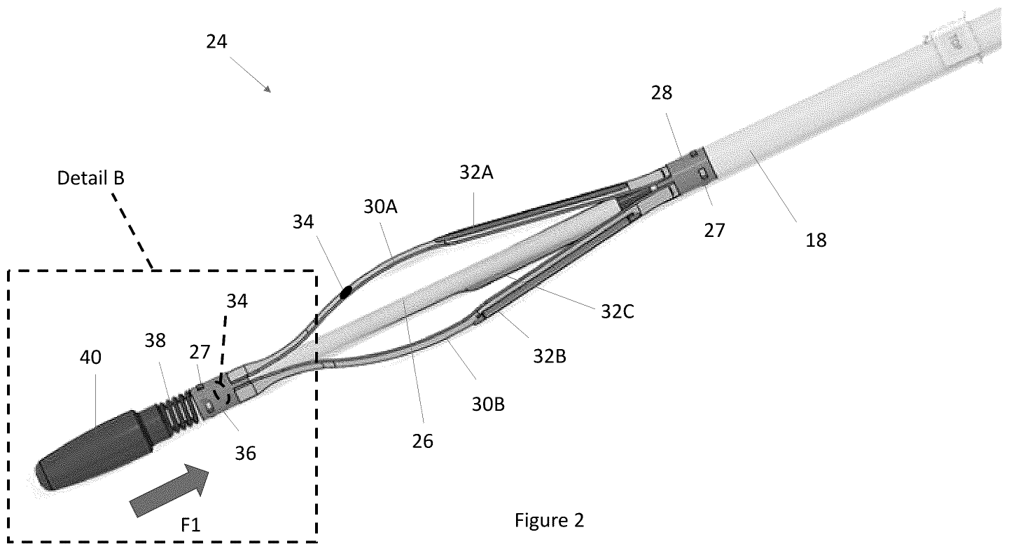

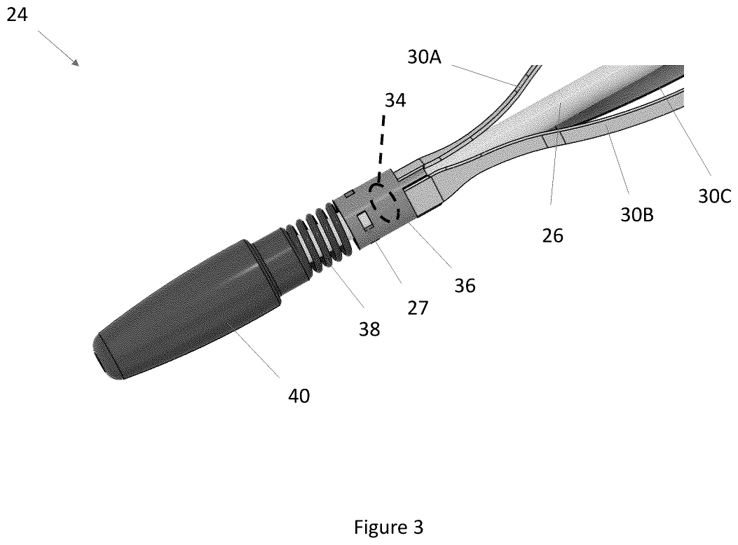

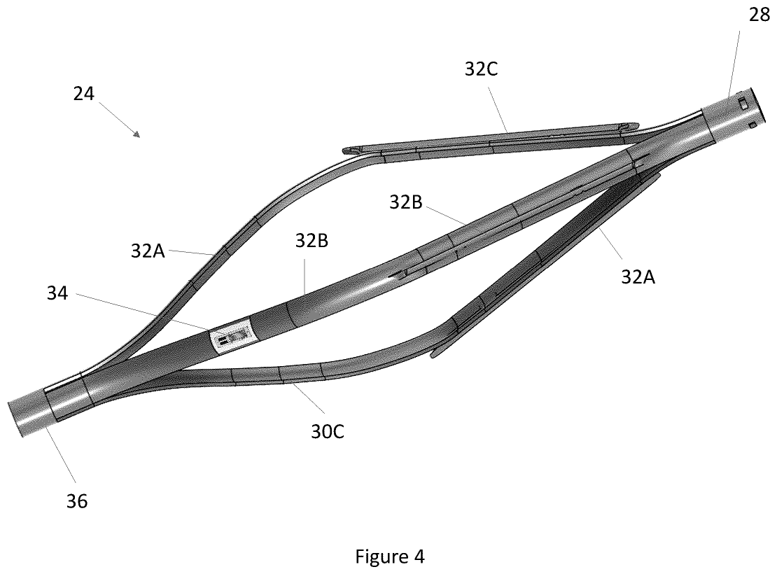

Referring additionally to FIG. 2 through FIG. 4, the expandable portion 24 may comprise one or more struts 30. In exemplary embodiments, without limitation, the expandable portion 24 comprises three struts 30A, 30B, 30C. The struts 30A, 30B, 30C may be provided substantially equidistant about the inner sleeve 26. Any number of struts 30 in any arrangement may be utilized. The struts 30, in exemplary embodiments without limitation, may each comprise a longitudinal axis that extends along a longitudinal axis of the expandable portion 24. The struts 30 may extend along a longitudinal axis of at least an adjacent portion of the guide wire 12, the inner sleeve 26, the catheter tube 18, and/or the sheath 20 in exemplary embodiments, without limitation. The struts 30 in exemplary embodiments, without limitation, may comprise a flattened outer surface. For example, without limitation, the struts 30 may comprise an oval or rectangular shaped cross section, though any size, shape, or kind of struts 30 may be utilized. This may permit the outer surfaces of the struts 30 to ride along tissue within the blood vessel and/or the blood vessel wall.

The expandable portion 24 may comprise a first common attachment component 28 and/or a second common attachment component 36. The struts 30 may be attached to the first common attachment component 28 at a first end thereof, and/or the second common attachment component 36 at a second end thereof. The inner sleeve 26 may be connected to the second common attachment component 36 in exemplary embodiments, without limitation. The first common attachment component 28 may be affixed to a distal end of the catheter tube 18 in exemplary embodiments. As the inner sleeve 26 may be configured for sliding movement while the catheter tube 18 remains relatively fixed, the struts 30 may be configured to bow outwardly upon retraction of the inner sleeve 26 in exemplary embodiments, such as a result of compressive force(s) F1 exerted against the struts 30. In exemplary embodiments, without limitation, the control element(s) 16 may comprise a slider which is operable for sliding movement within a slot 21 on the handle subassembly 14 to cause direct, translational sliding movement of the inner sleeve 26 within the catheter tube 18 to provide the compressive force(s) F1 and resulting movement of the struts 30 into an expanded position. In the expanded position (see e.g., FIGS. 2-5), a mid-portion of the struts 30 may bow outwardly away from the inner sleeve 26 such that the expandable portion 24 defines a maximum outer diameter, which is larger than a maximum outer diameter of the expandable portion 24 when in a collapsed position (see e.g., FIG. 1) where the struts 30 rest on, or extend along (such as but not limited to substantially parallel to) but are spaced apart from, the inner sleeve 26.

The first common attachment component 28, the second common attachment component 36, and/or the struts 30 may be integrally formed in exemplary embodiments. For example, without limitation, the first common attachment component 28, the second common attachment component 36, and/or the struts 30 may be formed in a sheet of material. Elongated slits may be formed, such as by punching, cutting, combinations thereof, or the like, into the sheet of material to form the struts 30 and/or the first and/or second common attachment components 28, 36 and rolled. Multiple such slits may be provided to form multiple struts 30A, 30B. Any number of slits and accompanying struts 30 may be formed at any arrangement or spacing. Some or all of the expandable portion 24 may be formed and/or assembled as provided in the Prior Disclosures.

The inner sleeve 26 may comprise components, such as but not limited to protrusions, configured to interact with components, such as but not limited to apertures 27, at the first common attachment component 28. Alternatively, or additionally, the catheter tube 18 may comprise components, such as but not limited to protrusions, configured to interact with components, such as but not limited to apertures 27, at the second common attachment component 36. Any number, size, shape, arrangement, or the like of such apertures 27 may be utilized.

In other exemplary embodiments, without limitation, the struts 30 may be joined to the first and/or second common attachment components 28, 36, such as by adhesive, welding, combinations thereof, or the like. The first and/or second common attachment components 28, 36 may be attached to the catheter tube 18 and the inner sleeve 26, respectively such as by protrusions on the catheter tube 18 and the inner sleeve 26 which frictionally engage with holes 27 in the first and/or second common attachment components 28, 36, respectively. Alternatively, or additionally, the first and/or second common attachment components 28, 36 may be attached to the catheter tube 18 and the inner sleeve 26 by adhesive, welding, combinations thereof, or the like.

The struts 30 may comprise one or more resiliently deformable materials such that the struts 30 are biased in the collapsed position, though such is not required. Alternatively, the struts 30 may be biased in the expanded position such that they are automatically expanded upon removal from the sheath 20. The struts 30 may comprise material which permits flexibility in bending to form an arch or other curve shape and bow outward. Alternatively, or additionally, the struts 30 may comprise one or more weakened regions, hinging areas, or the like which permit sections of the struts 30 to remain relatively non-deformed, at least along sections thereof. In this manner, proximal, medial, and/or distal portions of the struts 30 may form a relatively linear outer surface.

A tip member 40 may be provided. The tip member 40 may be connected to the second common connection component 36 and/or the inner sleeve 26. For example, the tip member 40 may be attached to the inner sleeve 26 at a position spaced apart from the second common connection component 36.

A limiter 38 may be provide between the second common connection component 36 and the tip member 40. Alternatively, the limiter 38 may be provided between the struts 30 and the tip member 40. The limiter 38 may be configured to cause the struts 30 to expand only to a predetermined size when placed in the expanded position. The limiter 38 may comprise one or more springs configured to provide sufficient forces (e.g., F1) axially along said inner sleeve 26 and/or catheter tube 18 towards said handle assembly 14 to force said struts 30 to bow outwardly when said struts 30 are exposed form the sheath 20 for expansion into the expanded position. In this way, the limiter 38 may act to bias the expandable portion 24 in the expanded position. Because the amount of force applied by the limiter 38 may be predetermined and/or limited, this may prevent the struts 30, and attached tissue modification elements 32, from expanding beyond a desired diameter and/or providing more forces or pressure than desired. This arrangement may permit retraction of the sheath 20 beyond the expandable portion 24 without necessarily changing the size of the expandable portion 24 and/or the forces exerted by the tissue modification elements 32. This may be particularly advantageous where the otherwise tortuous nature of the vascular system and/or other characteristics of the access site may make precise control of the size of the expandable portion 24, such as by movement of the control element(s) 16 difficult. This may also prevent over expansion of the expandable portion 24 and/or over exertion of forces at the tissue modification elements 32. The limiter 38 is not required. The tip member 40 may be configured to accommodate the guide wire 12.

In other exemplary embodiments, without limitation, the tip member 40 is located along the inner sleeve 26 at an area proximal to a proximal end of the struts 30, and the proximal end of the struts 30 are free such that the struts 30 are forced for bow outwardly as the tip member 40 is retracted along interior surfaces of the struts 30, such as in a cantilevered fashion. In such embodiments, a second end of the struts 30 may be free, such that the second common connection component 36 is not required. The struts 30 in such embodiments may be configured for cantilevered operation such as provided in the Prior Disclosures.

The sheath 20, in exemplary embodiments without limitation, may be configured for sliding movement so as to selectively expose, or cover, the expandable portion 24. One or more control element(s) 16 may be provided on the handle subassembly 14 for moving the sheath 20, though such is not required. For example, without limitation, the sheath 20 may be manually slidable. In other exemplary embodiments, without limitation, the sheath 20 may be fixed, and the expandable portion 24 may be moveable relative to the sheath 20.

Some or all of the struts 30 may comprise one or more of the tissue modification elements 32. Each of the tissue modification elements 32 may comprise a protrusion, blade, sharpened edge, blunted edge, combination thereof, or the like which extends from an outer surface of a respective one of the struts 30. Each of the tissue modification element 32 may extend along a longitudinal axis of the respective one of the struts 30 to which it is attached or forms part of. Each of the tissue modification elements 32 may extend along a longitudinal axis of the expandable portion 24. Each of the tissue modification elements 32 may extend along some, or all, of the respective one of the struts 30 which it is provided on. In exemplary embodiments, without limitation, the tissue modification elements 32 may extend along a proximal portion of the struts 30, such as but not limited to along substantially half, or less than half, of the struts 30. Each of the tissue modification elements 32 may comprise an arcuate element, a blunted cuboid protrusion, a triangular prism, combinations thereof, or the like to name a few examples without limitation. Any size, shape, or type of the tissue modification elements 32 may be utilized to score, incise, cut, remove, or otherwise modify atherosclerotic material or other tissue or elements at a blood vessel or other treatment area.

The intravascular device 10 may comprise one or more feedback devices 34. The feedback devices 34 may comprise one or more pressure sensors, hardness sensors, strain gauges, combinations thereof, or the like. One or more such feedback device 34 may be provided at exterior surfaces of said intravascular device 10, such as but not limited to along a portion of one or more of the struts 30. For example, without limitation, a feedback device 34 may be provided along a forward portion of a strut 30, at middle portion thereof, a proximal portion thereof, combinations thereof, or the like. The feedback devices 34 located along the strut(s) 30 may comprise hardness sensors for detecting hardness of surrounding tissue, pressure sensors for detecting pressure applied by strut(s) 30, strain gauges to measure strut 30 deflection, combinations thereof, or the like.

Alternatively, or additionally, one or more such feedback devices 34 may be provided at or between the second common attachment component 36, the limiter 38, and or the tip member 40. In this manner, the forces F1 provided, for example, may be determined.

A single or multiple such feedback devices 34 of the same or different type may be provided at some or all components of the expandable portion 24 to measure the same or different data points.

In exemplary embodiments, without limitation, the feedback device(s) 34 may comprise one or more strain gauges provided at one or more of the struts 30. The strain gauge(s) may be configured to measure strut 30 deflection, for example without limitation. For example, without limitation, the stain gauge(s) may be configured to measure localized deflection of a portion of an associated one of the struts 30. Alternatively, or additionally, the strain gauge(s) may be configured to measure forces exerted by the strut(s) 30 to the surrounding tissue. The strain measurements may be used to determine a level of resistance met at the tissue in exemplary embodiments, without limitation. This may reflect tissue hardness, operator force provided, luminal gain achieved, combinations thereof, or the like.

Alternatively, or additionally, the feedback device(s) 34 may comprise one or more pressure sensors. For example, the pressure sensor(s) may be provided at one or more of the struts 30 and may be configured to measure pressure exerted between one or more of the struts 30 and the tissue. As another example, without limitation, the pressure sensors may be located at, or between, the tip member 40, the limiter 38, and/or the second common attachment component 36, to measure compressive forces exerted to the struts 30. Regardless, the pressure measurements may be used to determine operator force, resistance encountered, luminal gain achieved, combinations thereof, or the like by way of non-limiting example. In exemplary embodiments, the pressure sensor(s) of the feedback device(s) may be attached to, or otherwise connected with, a spring or other component of the limiter 38.

Alternatively, or additionally still, the feedback device(s) 34 may comprise hardness sensors configured to measure hardness of the surrounding tissue. Such hardness sensors may include, for example without limitation, durometers, sclerometers, indenters, scleroscopes, pressure sensors, combinations thereof, or the like. Any type or kind of sensor, or combination of sensors, for measuring hardness of material, particularly that of human tissue, may be utilized.

Feedback data from the feedback device(s) 34 may be provided quantitively in raw form and/or qualitatively and/or quantitively as an associated exerted pressure, measure of expansion of the expandable portion 24, resistance encountered, operator force provided (e.g., at the expandable portion 24), luminal gain achieved, combinations thereof or the like. Such translational measurements may be made by way of one or more controller(s) 19 in exemplary embodiments. For example, without limitation, the controller(s) 19 may be configured to translate a raw measurement into a readable number and/or a graphical display approximating the reading relative to one or more benchmarks (e.g., graph, dial, gauge, bar chart, color coded, combinations thereof, or the like).

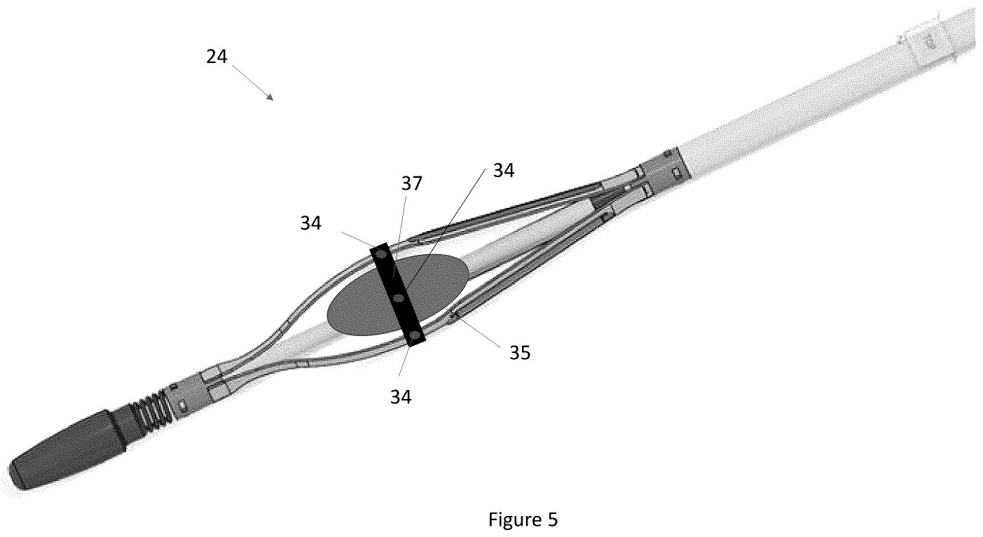

As illustrated in FIG. 5, one or more balloons 35 may be used as an alternative to, or addition to, the inner sleeve 26 for controlling movement of the struts 30. The balloon(s) 35 may be in fluid communication with a pump, reservoir, or compressed air source, combinations thereof, or the like at the handle subassembly 14 or outside thereof for providing inflation fluid to the balloon(s) 35. Fluid communication may be accomplished, in exemplary embodiments, by way of the catheter tube 18, the inner sleeve 26, and/or one or more tubes extending within the same. In such embodiments, the balloon(s) 35 may be inflated to about 1 atmosphere to 10 atmospheres. The inner sleeve 26 in such embodiments may be moveable, or fixed, such as to provide support to the balloon(s) 35. Alternatively, the inner sleeve 26 may not be utilized in such embodiments.

The feedback devices 34 may be provided on a ring or jacket 37 which may surround at least part of an outer surface of an expandable portion 24 of the intravascular device 10. The ring or jacket 34 may surround part or all of an outer surface of a balloon 35 and/or one or more struts 30 in exemplary embodiments without limitation. In other exemplary embodiments, without limitation, the feedback devices may be provided along an outer surface of, or integrated with, the balloon 35 or the struts 30 without the need for a separate ring or jacket 37.

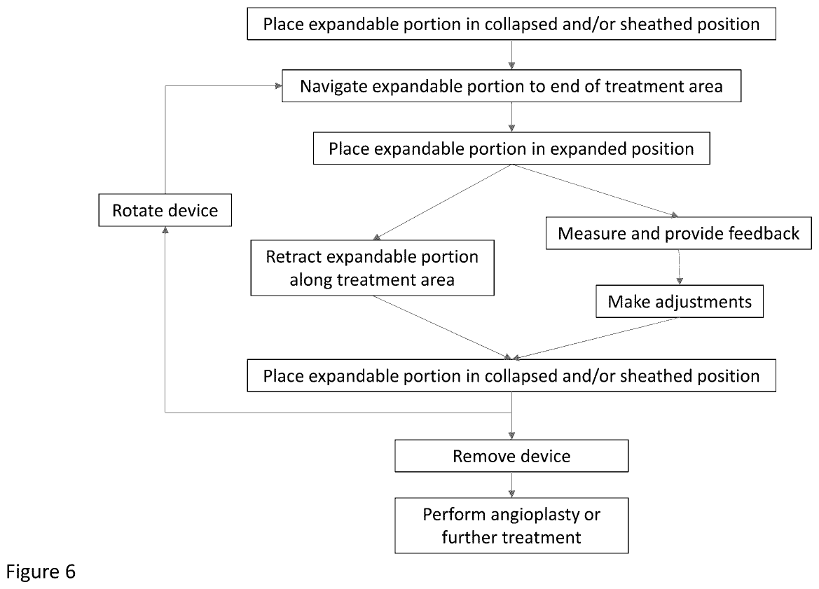

Referring additionally to FIG. 6, in exemplary embodiments, without limitation, the guide wire 12 may be introduced to the patient's vascular system and advanced to a treatment area. The guide wire 12 may be advanced to a distal end of the treatment area, or beyond to give room for maneuvering. Part of the device 10, such as the expandable portion 24, may be introduced to the patient's vascular system in the collapsed and/or sheathed state and advanced in the collapsed and/or sheathed state to a treatment area, such as an area of stenosis (e.g., plaque or other tissue accumulation) within a blood vessel. The treatment area may be a peripheral artery or fistula, for example without limitation-any location, or multiple locations, within the patient's vascular system may be treated. In exemplary embodiments, part of the device 10, such as the expandable portion 24, may be advanced along the guide wire 12. For example, without limitation, the expandable portion 24 may be manually fed over the guide wire 12 which may extend through the inner sleeve 26 and out the handle subassembly 14. Alternatively, or additionally, a distal portion of the inner sleeve 26 may comprise one or more holes, slits, or the like for allowing the guide wire 12 to enter and exit a portion of the expandable portion 24 without necessarily extending through all of the inner sleeve 26 and/or the handle subassembly 14.

The expandable portion 24 may be unsheathed and/or placed in the expanded position at an end of the treatment area and moved along the treatment area. The expandable portion 24 may be moved along the guide wire 12 extending therethrough. The expandable portion 24, and particularly the tissue modification elements 32, may modify the plaque or other tissue, such as by scoring, as the expandable portion 24 is retracted along the treatment area. The expandable portion 24 may be initially positioned at a distal end of the treatment area and retracted therethrough, such as along the guide wire 12, while in the expanded position to score tissue located therein. This may permit movement of the expandable portion 24 counter to natural blood flow, though such is not required. Scoring may include creation of axially extending slits in the plaque or other tissue. Doing so may break surface tension in the plaque or other tissue, resulting in luminal gain by itself and/or in combination with subsequent angioplasty, such as performed by a separate device at the treatment area once the expandable portion 24 is removed. Subsequent angioplasty is not required. Other treatments may also be simultaneously or subsequently performed, including but not limited to, imaging, stenting, medication delivery, combinations thereof, or the like. In exemplary embodiments, without limitation, the outer surface of the struts 30 may ride along the plaque or other tissue, such as while the expandable portion 24 is retracted. This may limit a penetrative depth of the tissue modification elements 32 into the plaque or other tissue.

The tissue modification elements 32 may be coated with one or more medications, in exemplary embodiments, without limitation. In this manner, medication may be delivered at the time of incision.

Multiple passes of the expandable portion 24 may be made, though a single pass may be utilized. The expandable portion 24 may be rotated between such passes to create additional slits, incisions, or other modifications to the plaque or other tissue. Such rotation may be performed at various times, including before repositioning the expandable portion 24 at the distal end of the treatment area, after repositioning the expandable portion 24 at the distal end of the treatment area, while repositioning the expandable portion 24 at the distal end of the treatment area, while retracting the expandable portion 24, combinations thereof, or the like. The expandable portion 24 need not be repositioned at the same start or end point for each retraction pass. Multiple passes may be made without rotation, such as to create deeper slits, wider slits, additional slits in close proximity, break through harder tissue, with different pressures or forces applied, different levels of expansion provided at the expandable portion 24, combinations thereof, or the like. Other types or kinds of tissue modifications may be achieved, such as with different size and/or shape tissue modification elements 32.

The feedback devices 34 may be in electronic communication with one or more feedback displays 15. Such electronic communication may be made by way of wired and/or wireless connections. Wires, near field communication devices, network connectivity devices, wireless routers, combinations thereof, or the like may be provided at the intravascular device 10, the feedback display 15, and/or interim components (e.g., servers, gateways, routers, remote devices, processors, controllers, etc.) for accomplishing the wired or wireless connection. Wires may be extended within or along the inner sleeve 26, catheter tube 18, sheath 20, combinations thereof, or the like in exemplary embodiments. Alternatively, or additionally, a wireless transmitter/receiver may be located at the expandable portion 24 for wireless communication.

The feedback displays 15 may be provided at the intravascular device 10, such as but not limited to, integrated with the handle subassembly 14, or at separate devices. The feedback display 15 may comprise electronic displays, gauge, dials, combinations thereof, or the like. The separate devices may comprise, but are not limited to, dedicated displays, computers, smartphone, tablets, smartwatches, combinations thereof, or the like. The feedback displays 15 may be configured to provide feedback data in substantially real-time or later. Such feedback data may comprise hardness and/or pressure readings from the feedback devices 34 by way of non-limiting example. The feedback data provided may be qualitative or quantitative, such as in absolute measure, relative measure, combinations thereof, or the like.

A user may monitor the feedback data provided at the feedback display 15 and adjust expansion of the expandable portion 24 accordingly. For example, without limitation, where harder atherosclerotic material is encountered, pressure exerted by the expandable portion 24 may be increased to a relatively higher level. As another example, again without limitation, where softer atherosclerotic material is encountered, pressure exerted by the expandable portion 24 may be decreased to a relatively lower level. In such embodiments, one or both or hardness, pressure, and/or strut 30 deflection may be measured and monitored, for example. In other exemplary embodiments, pressure exerted by the expandable portion 24 and/or deflection of the struts 30 may be adjusted to one or more benchmarks, thresholds, ranges, or the like. Such benchmarks, thresholds, ranges, or the like may be those known to result in minimal or acceptable levels of dissection, provide acceptable, desirable, or particular medical effects, combinations thereof, or the like. Such benchmarks, thresholds, ranges, or the like may be specific to hardness levels of the atherosclerotic material or not. In this way, chances of dissection may be minimized. Such benchmarks, thresholds, ranges, or the like may be provided at the feedback display 15, such as by way of one or more controllers 19 and/or in comparison with actual provided pressures. The control element(s) 16 in exemplary embodiments, without limitation, may be manually or automatically, such as by the controller 19, adjusted based on actual readings from the feedback device(s) 34 relative to such benchmarks, thresholds, ranges, or the like. The controller(s) 19 may be remote from the intravascular device 10 and/or integrated therewith.

While multiple feedback devices 34 are discussed in some places, a single feedback device for each intravascular device may be utilized of a same or different type. Where the intravascular device comprises one or more tissue modification elements 32, at least one of the feedback devices 34 may be positioned adjacent to one or more of the tissue modification elements 32, or on another strut 30 not comprising the tissue modification element 32, though any location may be utilized. Where multiple feedback devices 34 are utilized, the controller 19 may be configured to display multiple data points (e.g., one for each feedback device 34), provide an average reading, maximum reading, minimum reading, median reading, mode reading, combinations thereof, or the like.

After completing treatment, the expandable portion 24 may be removed from the treatment area and/or from the patient. Angioplasty may subsequently be performed, such as by way of one or more separate devices, though such is not required. Alternatively, or additionally, stents or other devices may be placed to help maintain the achieved luminal gains or other medical results. Such stents or other devices may be placed by the device 10, angioplasty device, or other device.

The struts 30, the tissue modification elements 32, and/or the balloon 35 may be coated with one or more materials, though such is not required. Such materials may comprise lubricious and/or medicated coatings in exemplary embodiments, without limitation.

Components of the device 10 may comprise one or more materials such as, but not limited to, one or more metals (e.g., nickel titanium alloy), polymers, combinations thereof, or the like. Materials utilized may be biocompatible, sterilizable, combinations thereof, or the like. Components of the device 10 may be integrally formed or joined, such as by welding, brazing, adhesion, mechanical fasteners, combinations thereof or the like.

While sometimes described as being used for treating plaque in blood vessels, such as sometimes results from peripheral artery disease, the system 11 and/or device 10 may be used to treat any type of kind of tissue in any location, such as but not limited to other bodily tissue within a patient's vascular system. For example, without limitation, the system 11 and/or device 10 may be used to treat stenosed areas of fistulas.

Any embodiment of the present invention may include any of the features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention.

Certain operations described herein may be performed by one or more electronic devices. Each electronic device may comprise one or more processors, electronic storage devices, executable software instructions, combinations thereof, and the like configured to perform the operations described herein. The electronic devices may be general purpose computers or specialized computing devices. The electronic devices may comprise personal computers, smartphone, tablets, databases, servers, or the like. The electronic connections and transmissions described herein may be accomplished by wired or wireless means. The computerized hardware, software, components, systems, steps, methods, and/or processes described herein may serve to improve the speed of the computerized hardware, software, systems, steps, methods, and/or processes described herein.

What is claimed is:

1. An intravascular device comprising:
an expandable portion comprising a plurality of struts movable between a first position and a second position where at least a portion of each of the struts is moved outward relative to the first position;
one or more tissue modification elements, each provided at a respective one of the plurality of struts;
a tip member located distal to, and spaced apart from, a distal end of the expandable portion; and
a spring interposed between said expandable portion and said tip member.

2. The intravascular device of claim 1 further comprising:
an inner sleeve extending through the expandable portion to said tip member such that retraction of said inner sleeve causes movement of the struts into the second position.

3. The intravascular device of claim 1 further comprising:
a balloon located between the struts, which when inflated, moves the struts into the second position.

4. The intravascular device of claim 3 further comprising:
a fluid passageway within the catheter tube fluidly connected to the balloon for receiving inflation fluid for moving the balloon into an inflated state, thereby moving the struts into the second position.

5. The intravascular device of claim 1 further comprising:
a catheter tube having a distal end affixed to a proximal end of the expandable portion; and
a handle assembly, wherein a proximal end portion of said catheter tube extends from said handle assembly.

6. The intravascular device of claim 1 wherein:
said expandable portion includes a first common attachment component and a second common attachment component;
each of the struts extends longitudinally between the first and second common attachment components;
at least a mid-portion of the struts bows outwardly when in the second position.

7. The intravascular device of claim 6 wherein:
a longitudinal axis of each of the one or more including elements extends along a longitudinal axis of the respective one of the struts, at least while in the first position.

8. The intravascular device of claim 7 wherein:
each of the one or more tissue modification elements comprise a cuboid shaped protrusion extending along a proximal half of the respective one of the struts and outward therefrom.

9. The intravascular device of claim 1 wherein:
said spring comprises a coil spring having a plurality of coils extending about a portion of said inner sleeve.

10. The intravascular device of claim 1 further comprising:
at least one sensor connected to at least one of: said spring, said tip member, and said expandable portion for measuring forces experienced at said spring.

11. The intravascular device of claim 10 wherein:
said at least one sensor comprises a pressure sensor.

12. The intravascular device of claim 1 further comprising:
one or more sensors provided at the struts.

13. The intravascular device of claim 12 wherein:
said one or more sensors comprise a strain gauge or a hardness sensor.

14. The intravascular device of claim 1 wherein:
at least one of the one or more tissue modification elements is provided at, and extends from, an outer surface of the respective one of the struts.

15. The intravascular device of claim 1 wherein:
the one or more tissue modification elements comprise a plurality of tissue modification elements, at least one of which is provided at an outer surface of each respective one of the struts.

16. The intravascular device of claim 1 further comprising:
a sheath movable over the expandable portion.

17. The intravascular device of claim 1 further comprising:
one or more feedback devices provided at the expandable portion, said one or more feedback devices selected from the group consisting of: durometers, sclerometers, indenters, scleroscopes, pressure sensors, strain gauges, and accelerometers, wherein said one or more feedback devices are in wired or wireless electronic communication with a feedback display.

18. An intravascular device comprising:
an expandable portion comprising a plurality of struts selectively movable between a first position and a second position where at least a portion of each of the struts is moved outward relative to the first position;
one or more tissue modification elements, each provided at a respective one of the plurality of struts; and
a balloon located between the struts, which when inflated, moves the struts into the second position.

19. The intravascular device of claim 18 further comprising:
a tip member located distal to, and spaced apart from, a distal end of the expandable portion; and
a spring interposed between said expandable portion and said tip member.

20. An intravascular device comprising:
a handle assembly;
a catheter tube having a proximal end portion extending from said handle assembly;
an expandable portion having a proximal end affixed to a distal end of the catheter tube, said expandable portion comprising a first common attachment portion, a second common attachment portion, and a plurality of struts extending longitudinally between the first and second common attachment portions, said struts movable between a first position and a second position where at least a mid-portion of each of the struts is bowed outward relative to the first position;
tissue modification elements, each comprising a cuboid shaped protrusion provided at, and extending outward from, an outer surface of a proximal portion of a respective one of the struts;
a tip member located distal to, and spaced apart from, a distal end of the expandable portion;
an inner sleeve extending through the expandable portion to said tip member such that retraction of said inner sleeve causes movement of the struts into the second position;
a coil spring interposed between said expandable portion and said tip member and extending about a portion of said inner sleeve; and
a sheath movable over the expandable portion.
